(19) United States
(12) Patent Application Publication
(10) Pub. No.: US 2014/0350256 A1
(43) Pub. Date: Nov. 27, 2014

(54) IRON AND MANGANESE COMPLEXES COMPRISING HEXADENTATE MONOCARBOXYLATO-CONTAINING LIGANDS AND THEIR USE FOR THE CATALYSIS OF OXIDATION REACTIONS

(71) Applicant: University of Southern Denmark, Odense M (DK)

(72) Inventor: Christine MCKENZIE, Odense (DK)

(73) Assignee: UNIVERSITY OF SOUTHERN DENMARK, Odense M (DK)

(21) Appl. No.: 14/282,279

(22) Filed: May 20, 2014

Related U.S. Application Data
(60) Provisional application No. 61/825,549, filed on May 21, 2013.

(51) Int. Cl. B01J 31/22; C07F 13/00; C07D 301/03; C07C 315/02; C01B 3/06; C01B 13/02; C07F 15/02; C07B 33/00

(52) U.S. Cl. CPC B01J 31/2295; C07F 15/025; C07F 13/005; C07B 33/00; C07C 315/02; C01B 3/06; C01B 13/0207; C07D 301/03; B01J 2231/72; B01J 2231/70; C01B 2203/1088; C01B 2203/1047
USPC 546/5

(57) ABSTRACT

The present invention relates to iron and manganese complexes based on hexadentate ligand systems containing one carboxylato donor and five nitrogen donor atoms and to their use in methods for the catalysis of the oxidation of organic substrates. The manganese complexes of the invention also may be used as (pro)-catalysts in methods for the catalysis of water.

### CROSS-REFERENCE TO RELATED APPLICATION

This application claims priority to U.S. Provisional Patent Application Ser. No. 61/825,549, filed on May 21, 2013, the contents of which are incorporated herein by reference.

### FIELD OF THE INVENTION

The present invention relates to iron and manganese complexes of hexadentate ligands containing one carboxylato donor group and to their use for the catalysis of oxidation reactions. In particular, the invention relates to iron and manganese complexes based on hexadentate ligand systems containing one carboxylato donor and five nitrogen donor atoms and to their use in the generation of catalytically competent oxidants for the catalysis of oxidation reactions.

### BACKGROUND OF THE INVENTION

The role of iron in the enzymatic oxidation of organic substrates with molecular oxygen is of central importance in aerobic life. These reactions are of great industrial interest for replacing the use of toxic stoichiometric oxidants, such as chromate, in the synthesis of a wide range of fine chemicals, including drugs. One approach is to use synthetic molecular catalysts based on inexpensive, non-toxic metals such as iron, wherein metal-promoted oxidations of coordinated ligands in such synthetic metal complexes mimic the reactions catalyzed by metalloenzymes involved in the oxidation of substrates using oxygen or its activated derivatives.

Similarly, manganese is involved in biological redox reactions involving water, peroxide and oxygen where oxidation states II, III and IV are implicated in mono-, di- and multi-nuclear centers. These include, for example, the oxygen evolving center (OEC) of photosystem II, catalases, manganese ribonucleotide reductase, superoxide dismutases, and peroxidases.

The synthesis of manganese complexes which can mimic biological processes and, in particular, promote the oxidation of water, is considered to have significant implications for technologies in the area of renewable energy.

WO-A-9534628 describes the use of iron complexes containing certain pentadentate nitrogen-containing ligands, in particular, N,N-bis(pyridin-2-yl-methyl)-bis(pyridin-2-yl)methylamine [“N4Py”], as bleaching and oxidation catalysts.

WO-A-9718035 discloses iron and manganese complexes containing tetra-, penta- and hexacoordinating nitrogenated ligands such as N,N′-bis(pyridine-2-yl-methyl)ethylene-1,2-diamine [“Bispicen”], N-methyl-N,N′,N′-tris(pyridine-2-yl-methyl)ethylene-1,2-diamine [“TrispicMeen”], and N,N,N′,N′-tetrakis(pyridine-2-yl-methyl)ethylene-1,2-diamine [“TPEN”] as peroxide oxidation catalysts for organic substrates.

U.S. Pat. No. 6,165,963 discloses compounds having a pentadentate ligand comprising nitrogen-donor containing heteroaryl groups for use with peroxygen bleaching agents.

Various iron and manganese complexes having pentadentate ligands such as N-carboxomethyl-N′—R—N,N′-bis(2-pyridylmethyl)-1,2-ethandiamine (R=methyl or benzyl) have been described in J. Chem. Soc., Dalton Trans., 2003, 1765-1772 and in Angew. Chem. Int. Ed. 2006, 45, 1602-1606. The manganese complexes are indicated for the catalytic oxidation of water in J. Chem. Soc., Dalton Trans., 2011, 40, 3849-3858 and 3859-3870.

Whilst known iron and manganese complexes have to an extent been used successfully as oxidation catalysts, there remains a need for other such complexes that are more effective in terms of activity.

The present invention is based, in part, on the fact that catalytically highly active iron and manganese complexes based on hexadentate ligand systems containing one carboxylato donor and five nitrogen donor atoms surprisingly can activate a range of oxidants and thereby, act as oxidation catalysts for a wide range of substrates.

### SUMMARY OF THE INVENTION

In one aspect, the present invention provides iron and manganese complexes having the following general formulas (I), (II) and (III):

[(LH)₂Fe₂(O)]^{z}Y_{q} (or its deprotonated form: [(L)₂Fe₂(O)]^{z}Y_{q}) (I)

[LM]^{z}Y_{q} (II)

[LM(X)]^{z}Y_{q} (III)

wherein:

M represents a metal ion selected from Fe or Mn in the II, III, IV or V oxidation state; L represents a monoanionic potentially hexadentate ligand containing one carboxylate group and five nitrogen donor atoms, which ligand coordinates to the Fe or Mn metal ion in the complex via said carboxylate group and at least four of said nitrogen donor atoms and has the general formula (IV):

R¹R¹N-A-N(R¹)WC(O)O⁻ (IV)

wherein:

each R¹ independently represents —R²—B, in which R² represents an optionally substituted group selected from the group consisting of alkylene, alkenylene, oxyalkylene, aminoalkylene and alkylene ether, and B represents an optionally substituted heteroaryl group selected from the group consisting of pyridinyl, pyrazinyl, pyrazolyl, imidazolyl, benzimidazolyl, pyrimidinyl, triazolyl and thiazolyl;

A represents an optionally substituted alkylene or arylene bridging group selected from the group consisting of —CH₂CH₂—, —CH₂CH₂CH₂—, —CH₁₀—, and —C₆H₄—; and

W represents a group selected from the group consisting of alkylene, arylene and arylalkylene;

X represents a coordinating species selected from the group consisting of oxygen-coordinating terminal oxidants including peroxides, peracids, iodosylaryls, hypohalides, oxone (peroxymonosulfate), N-oxides and dioxygen; H₂O, ROH, RCN, OH⁻, RO⁻, RCOO⁻, F⁻, Cl⁻, Br⁻, I⁻, O²⁻, NO₃⁻, NO₂⁻, ClO₄⁻; or aromatic nitrogen-donors selected from the group consisting of pyridines, pyrazines, pyrazoles, imidazoles, benzimidazoles, pyrimidines, triazoles and thiazoles, with R being selected from H, optionally substituted alkyl or optionally substituted aryl;

Y represents a non-coordinating counter ion;

z represents the charge of the complex and is an integer which can be positive or zero;

q=z/[charge Y].

The aforementioned iron and manganese complexes can be used as (pro)-catalysts in oxidation reactions to catalyze, for example, the oxidation of organic substrates and, in the case of the manganese complexes, to catalyze the oxidation of water.

Accordingly, in another aspect, the present invention provides a method for the catalysis of oxidation reactions, which method comprises combining an iron or manganese complex having formula (I), (II) or (III) as defined herein above with the compound to be oxidized in solution, together with an appropriate oxidant and, after completion of the oxidation reaction, isolating the oxidized product.

The aforementioned method of the invention may be employed for the catalysis of the oxidation of organic substrates, wherein an iron or manganese complex having formula (I), (II) or (III) as defined herein above is combined with the organic substrate which is to be oxidized in an organic solvent, together with an appropriate oxidant. The aforementioned method of the invention may be employed for the catalysis of the oxidation of water, wherein an appropriate oxidant is added to an aqueous solution of a manganese complex having formula (II) or (III) as defined herein above, which aqueous solution may optionally include an organic solvent.

In a yet a further aspect, the present invention provides a process for the preparation of reactive complex-oxidant adducts of formula (III) herein above, wherein the coordinating species X is an oxidant selected from peroxides, peracids, iodosylaryls, hypohalides, oxone, N-oxides or dioxygen, which process comprises the step of mixing [(LH)₂Fe₂(O)]^{z}Y_{q} according to formula (I) as defined herein above or [LM]^{z}Y_{q} according to formula (II) as defined herein above with an oxidant in an organic solvent, wherein the oxidant is selected from peroxides, peracids, iodosylaryls, hypohalides, oxone, N-oxides or dioxygen.

### DEFINITIONS

The term “optional” as used herein includes all conceivable variations. Compounds and groups which are “optionally” substituted accordingly embrace unsubstituted and substituted compounds and groups, respectively, for the purposes of the present invention. Examples of such substituents include, with out being limited thereto, alkyl; alkoxy; hydroxy; halides and aryl.

For the purposes of this invention the terms “alkyl, alkylene, alkyenylene, oxyalkylene, alkoxy, arylene, aryl, amine and halides” are, unless specified otherwise, as follows:

Alkyl=linear or branched C1-C8 alkyl.

Alkylene=C1-C8 alkylene. Particular preference is given to methylene; 1,1-ethylene; 1,2-ethylene; 1,1-propylidene; 1,2-propylene; 1,3-propylene; 2,2-propylidene; 1,4-butylene; cyclohexane-1,1-diyl; cyclohexane-1,2-diyl; cyclohexane-1,3-diyl; cyclohexane-1,4-diyl; cyclopentane-1,1-diyl; cyclopentane-1,2-diyl; and cyclopentane-1,3-diyl.

Alkyenylene=C2-C6 alkyenylene.

Oxyalkylene=C1-C6 oxyalkylene.

Alkoxy=C1-C6 alkoxy.

Arylene=1,2 phenylene; 1,3-phenylene; 1,4-phenylene; 1,2-naphthylene; 1,3-naphthylene; 1,4-naphthylene; 2,3-naphthylene.

Aryl=homoaromatics having a molecular weight of ≧300. Preference is particularly given to aryl groups selected from the group consisting of phenyl, biphenyl and naphthyl.

Amine=—N(R)₂ in which each R independently of the other is selected from the group consisting of: H; C1-C6 alkyl; C1-C6 alky-C₆H₅; and phenyl, it being possible for the two Rs to form a —NC3 to —NC5 heterocyclic ring closure.

Halide=F⁻; Cl⁻; Br⁻ and I⁻.

For the purposes of this invention the term “monoanionic ligand”, as used herein in relation to the ligand L containing one carboxylate group and five nitrogen donor atoms, encompasses such ligands wherein, when the iron or manganese complexes of the invention are in solution, any non-coordinated heteroaryl group of the ligand L may be protonated depending on the pH of the solution.

### DETAILED DESCRIPTION OF THE INVENTION

The iron and manganese complexes of the invention have the following general formulas (I), (II) and (III):

[(LH)₂Fe₂(O)]^{z}Y_{q} (or its deprotonated form: [(L)₂Fe₂(O)]^{z}Y_{q}) (I)

[LM]^{z}Y_{q} (II)

[LM(X)]^{z}Y_{q} (III)

wherein:

M represents a metal selected from Fe or Mn in the II, III, IV or V oxidation state;

L represents a monoanionic potentially hexadentate ligand containing one carboxylate group and five nitrogen donor atoms, which ligand coordinates to the Fe or Mn metal ion in the complex via said carboxylate group and at least four of said nitrogen donor atoms and has the general formula (IV):

R¹R¹N-A-N(R¹)W(O)O⁻ (IV)

wherein:

each R¹ independently represents R²—B, in which R² represents an optionally substituted group selected from the group consisting of alkylene, alkenylene, oxyalkylene, aminoalkylene and alkylene ether, and B represents an optionally substituted heteroaryl group selected from the group consisting of pyridinyl, pyrazinyl, pyrazolyl, imidazolyl, benzimidazolyl, pyrimidinyl, triazolyl and thiazolyl;

A represents an optionally substituted alkylene or arylene bridging group selected from the group consisting of CH₂CH₂—, —CH₂CH₂CH₂—, —C₆H₁₀—, and —C₆H₄—; and

W represents a group selected from the group consisting of alkylene, arylene and arylalkylene;

X represents a coordinating species selected from the group consisting of oxygen-coordinating terminal oxidants including peroxides, peracids, iodosylaryls, hypohalides, oxone, N-oxides and dioxygen; H₂O, ROH, RCN, OH⁻, RO⁻, RCOO⁻, F⁻, Cl⁻, Br⁻, I⁻, O²⁻, NO₃⁻, NO₂⁻, ClO₄⁻; or aromatic nitrogen-donors selected from the group consisting of pyridines, pyrazines, pyrazoles, imidazoles, benzimidazoles, pyrimidines, triazoles and thiazoles, with R being selected from H, optionally substituted alkyl or optionally substituted aryl;

Y represents a non-coordinating counter ion;

z represents the charge of the complex and is an integer which can be positive or zero;

q=z/[charge Y].

The ligand L having the general formula (IV) as indicated above is potentially a hexadentate ligand. The term ‘hexadentate’ in the sense of the present invention means that, in addition to the carboxylate group, five nitrogen atoms can coordinate to the iron or manganese metal ion in the metal complex. Accordingly, in formula (IV) at least one potentially coordinating nitrogen atom is present in each of the three R¹ groups.

The optionally substituted heteroaryl group B of the R¹ group(s) in formula (IV) is preferably a pyrindin-2-yl group. Possible substituents include C1-C4 alkyl, most preferably methyl; C1-C4 alkoxy, most preferably methoxy; and hydroxyl. The heteroaryl group is attached via a nitrogen atom in formula (IV), preferably via an alkylene group and, more preferably, via a methylene group. Most preferably, the heteroaryl group is a pyrindin-2-yl group which is attached to a nitrogen atom via methylene.

The bridging group A can be a substituted or unsubstituted alkylene or arylene group selected from the group consisting of CH₂CH₂—, —CH₂CH₂CH₂—, —C₆H₁₀—, and —C₆H₄—. Possible substituents include C1-C4 alkyl, most preferably methyl; C1-C4 alkoxy, most preferably methoxy; and hydroxyl. Preferably, the bridging group A is an ethylene group.

Examples of preferred ligands of the formula (IV) in their simplest (i.e., unsubstituted) forms are:
N,N,N′-tris(2-pyridylmethyl)ethylenediamine-N′-acetate;
N,N,N′-tris(2-pyridylmethyl)cyclohexanediamine-N′-acetate;
N,N,N′-tris(2-pyridylmethyl)isopropylenediamine-N′-acetate;
N,N,N′-tris(2-pyridylmethyl)ethylenediamine-N′-alanate;
N,N,N′-tris(2-pyridylmethyl)cyclohexanediamine-N′-alanate;
N,N,N′-tris(2-pyridylmethyl)isopropylenediamine-N′-alanate;
N,N,N′-tris(2-pyridylmethyl)ethylenediamine-N′-methylalanate;
N,N,N′-tris(2-pyridylmethyl)cyclohexanediamine-N′-methylalanate;
N,N,N′-tris(2-pyridylmethyl)isopropylenediamine-N′-methylalanate;
N,N,N′-tris(1-pyrazolylmethyl)ethylenediamine-N′-acetate;
N,N,N′-tris(1-pyrazolylmethyl)cyclohexanediamine-N′-acetate;
N,N,N′-tris(1-pyrazolylmethyl)isopropylenediamine-N′-acetate;
N,N,N′-tris(1-pyrazolylmethyl)ethylenediamine-N′-alanate;
N,N,N′-tris(1-pyrazolylmethyl)cyclohexanediamine-N′-alanate;
N,N,N′-tris(1-pyrazolylmethyl)isopropylenediamine-N′-alanate;
N,N,N′-tris(1-pyrazolylmethyl)ethylenediamine-N′-methylalanate;
N,N,N′-tris(1-pyrazolylmethyl)cyclohexanediamine-N′-methylalanate;
N,N,N′-tris(1-pyrazolylmethyl)isopropylenediamine-N′-methylalanate;
N,N,N′-tris(2-imidazolylmethyl)ethylenediamine-N′-acetate;
N,N,N′-tris(2-imidazolylmethyl)cyclohexanediamine-N′-acetate;
N,N,N′-tris(2-imidazolylmethyl)isopropylenediamine-N′-acetate;
N,N,N′-tris(2-imidazolylmethyl)ethylenediamine-N′-alanate;
N,N,N′-tris(2-imidazolylmethyl)cyclohexanediamine-N′-alanate;
N,N,N′-tris(2-imidazolylmethyl)isopropylenediamine-N′-alanate;
N,N,N′-tris(2-imidazolylmethyl)ethylenediamine-N′-methylalanate;
N,N,N′-tris(2-imidazolylmethyl)cyclohexanediamine-N′-methylalanate;
N,N,N′-tris(2-imidazolylmethyl)isopropylenediamine-N′-methylalanate;
N,N,N′-tris(2-pyrimidylmethyl)ethylenediamine-N′-acetate;
N,N,N′-tris(2-pyrimidylmethyl)ethylenediamine-N′-acetate;
N,N,N′-tris(2-pyrimidylmethyl)cyclohexanediamine-N′-acetate;
N,N,N′-tris(2-pyrimidylmethyl)isopropylenediamine-N′-acetate;
N,N,N′-tris(2-pyrimidylmethyl)ethylenediamine-N′-alanate;
N,N,N′-tris(2-pyrimidylmethyl)cyclohexanediamine-N′-alanate;
N,N,N′-tris(2-pyrimidylmethyl)isopropylenediamine-N′-alanate;
N,N,N′-tris(2-pyrimidylmethyl)ethylenediamine-N′-methylalanate;
N,N,N′-tris(2-pyrimidylmethyl)cyclohexanediamine-N′-methylalanate;
N,N,N′-tris(2-pyrimidylmethyl)isopropylenediamine-N′-methylalanate;
N,N,N′-tris(1-1,2,4-triazolylmethyl)ethylenediamine-N′-acetate;
N,N,N′-tris(1-1,2,4-triazolylmethyl)cyclohexanediamine-N′-acetate;
N,N,N′-tris(1-1,2,4-triazolylmethyl)isopropylenediamine-N′-acetate;
N,N,N′-tris(1-1,2,4-triazolylmethyl)ethylenediamine-N′-alanate;
N,N,N′-tris(1-1,2,4-triazolylmethyl)cyclohexanediamine-N′-alanate;
N,N,N′-tris(1-1,2,4-triazolylmethyl)isopropylenediamine-N′-alanate;
N,N,N′-tris(1-1,2,4-triazolylmethyl)ethylenediamine-N′-methylalanate;
N,N,N′-tris(1-1,2,4-triazolylmethyl)cyclohexanediamine-N′-methylalanate;
N,N,N′-tris(1-1,2,4-triazolylmethyl)isopropylenediamine-N′-methylalanate;
N,N-bis(2-pyridylmethyl)ethylenediamine-M-1-pyrazolylmethyl-N′-acetate;
N,N-bis(2-pyridylmethyl)cyclohexanediamine-N′-1-pyrazolylmethyl-N′-acetate;
N,N-bis(2-pyridylmethyl)isopropylenediamine-N′-1-pyrazolylmethyl-N′-acetate;
N,N-bis(2-pyridylmethyl)ethylenediamine-N′-1-pyrazolylmethyl-N′-alanate;
N,N-bis(2-pyridylmethyl)isopropylenediamine-N′-1-pyrazolylmethyl-N′-alanate;
N,N-bis(2-pyridylmethyl)isopropylenediamine-N′-1-pyrazolylmethyl-N′-alanate;
N,N-bis(2-pyridylmethyl)ethylenediamine-N′-1-pyrazolylmethyl-N′-methylalanate;
N,N-bis(2-pyridylmethyl)isopropylenediamine-N′-1-pyrazolylmethyl-N′-methylalanate;
N,N-bis(2-pyridylmethyl)isopropylenediamine-N′-1-pyrazolylmethyl-N′-methylalanate;
N,N-bis(2-pyridylmethyl)ethylenediamine-N′-2-imidazolylmethyl-N′-acetate;
N,N-bis(2-pyridylmethyl)cyclohexanediamine-N′-2-imidazolylmethyl-N′-acetate;
N,N-bis(2-pyridylmethyl)isopropylenediamine-N′-2-imidazolylmethyl-N′-acetate;
N,N-bis(2-pyridylmethyl)ethylenediamine-N′-1-2-imidazolylmethyl-N′-alanate;
N,N-bis(2-pyridylmethyl)cyclohexanediamine-N′-1-2-imidazolylmethyl-N′-alanate;
N,N-bis(2-pyridylmethyl)isopropylenediamine-N′-1-2-imidazolylmethyl-N′-alanate;
N,N-bis(2-pyridylmethyl)ethylenediamine-N′-1-2-imidazolylmethyl-N′-methylalanate;
N,N-bis(2-pyridylmethyl)cyclohexanediamine-N′-1-2-imidazolylmethyl-N′-methylalanate;
N,N-bis(2-pyridylmethyl)isopropylenediamine-N′-1-2-imidazolylmethyl-N′-methylalanate;
N,N-bis(2-pyridylmethyl)ethylenediamine-N′-2-pyrimidylmethyl-N′-acetate;
N,N-bis(2-pyridylmethyl)cyclohexanediamine-N′-2-pyrimidylmethyl-N′-acetate;
N,N-bis(2-pyridylmethyl)isopropylenediamine-N′-2-pyrimidylmethyl-N′-acetate;
N,N-bis(2-pyridylmethyl)ethylenediamine-N′-2-pyrimidylmethyl-N′-alanate;
N,N-bis(2-pyridylmethyl)cyclohexanediamine-N′-2-pyrimidylmethyl-N′-alanate;
N,N-bis(2-pyridylmethyl)isopropylenediamine-N′-2-pyrimidylmethyl-N′-alanate;
N,N-bis(2-pyridylmethyl)ethylenediamine-N′-2-pyrimidylmethyl-N′-methylalanate;
N,N-bis(2-pyridylmethyl)cyclohexanediamine-N′-2-pyrimidylmethyl-N′-methylalanate;
N,N-bis(2-pyridylmethyl)isopropylenediamine-N′-2-pyrimidylmethyl-N′-methylalanate;
N,N-bis(2-pyridylmethyl)ethylenediamine-N′-1-1,2,4-triazolylmethyl-N′-acetate;
N,N-bis(2-pyridylmethyl)cyclohexanediamine-N′-1,2,4-triazolylmethyl-N′-acetate;
N,N-bis(2-pyridylmethyl)isopropylenediamine-N′-1,2,4-triazolylmethyl-N′-acetate;
N,N-bis(2-pyridylmethyl)ethylenediamine-N′-1,2,4-triazolylmethyl-N′-alanate;
N,N-bis(2-pyridylmethyl)cyclohexanediamine-N′-1,2,4-triazolylmethyl-N′-alanate;
N,N-bis(2-pyridylmethyl)isopropylenediamine-N′-1,2,4-triazolylmethyl-N′-alanate;
N,N-bis(2-pyridylmethyl)ethylenediamine-N′-1,2,4-triazolylmethyl-N′-methylalanate;
N,N-bis(2-pyridylmethyl)cyclohexanediamine-N′-1,2,4-triazolylmethyl-N′-methylalanate;
N,N-bis(2-pyridylmethyl)isopropylenediamine-N′-1,2,4-triazolylmethyl-N′—N,N-bis(1-pyrazolylmethyl)ethylenediamine-N′-2-pyridylmethyl-N′-acetate;
N,N-bis(1-pyrazolylmethyl)cyclohexanediamine-N′-2-pyridylmethyl-N′-acetate;
N,N-bis(1-pyrazolylmethyl)isopropylenediamine-N′-2-pyridylmethyl-N′-acetate;
N,N-bis(1-pyrazolylmethyl)ethylendiamine-N′2-pyridylmethyl-N′-alanate;
N,N-bis(1-pyrazolylmethyl)cyclohexanediamine-N′-2-pyridylmethyl-N′-alanate;
N,N-bis(1-pyrazolylmethyl)isopropylenediamine-N′-2-pyridylmethyl-N′-alanate;
N,N-bis(1-pyrazolylmethyl)ethylendiamine-N′-2-pyridylmethyl-N′-methylalanate;
N,N-bis(1-pyrazolylmethyl)cyclohexanediamine-N′-2-pyridylmethyl-N′-methylalanate;
N,N-bis(1-pyrazolylmethyl)isopropylenediamine-N′-2-pyridylmethyl-N′-methylalanate;
N,N-bis(2-imidazolylmethyl)ethylenediamine-N′-2-pyridylmethyl-N′-acetate;
N,N-bis(2-imidazolylmethyl)cyclohexanediamine-N′-2-pyridylmethyl-N′-acetate;
N,N-bis(2-imidazolylmethyl)isopropylenediamine-N′-2-pyridylmethyl-N′-acetate;
N,N-bis(2-imidazolylmethyl)ethylendiamine-N′-2-pyridylmethyl-N′-alanate;
N,N-bis(2-imidazolylmethyl)cyclohexanediamine-N′-2-pyridylmethyl-N′-alanate;
N,N-bis(2-imidazolylmethyl)isopropylenediamine-N′-2-pyridylmethyl-N′-alanate;
N,N-bis(2-imidazolylmethyl)ethylendiamine-N′-2-pyridylmethyl-N′-methylalanate;
N,N-bis(2-imidazolylmethyl)cyclohexanediamine-N′-2-pyridylmethyl-N′-methylalanate;
N,N-bis(2-imidazolylmethyl)isopropylenediamine-N′-2-pyridylmethyl-N′-methylalanate;
N,N-bis(2-pyrimidylmethyl)ethylenediamine-N′-2-pyridylmethyl-N′-acetate;
N,N-bis(2-pyrimidylmethyl)cyclohexanediamine-N′-2-pyridylmethyl-N′-acetate;
N,N-bis(2-pyrimidylmethyl)isopropylenediamine-N′-2-pyridylmethyl-N′-acetate;
N,N-bis(2-pyrimidylmethyl)ethylendiamine-N′-2-pyridylmethyl-N′-alanate;
N,N-bis(2-pyrimidylmethyl)cyclohexanediamine-N′-2-pyridylmethyl-N′-alanate;
N,N-bis(2-pyrimidylmethyl)isopropylenediamine-N′-2-pyridylmethyl-N′-alanate;
N,N-bis(2-pyrimidylmethyl)ethylendiamine-N′-2-pyridylmethyl-N′-methylalanate;
N,N-bis(2-pyrimidylmethyl)cyclohexanediamine-N′-2-pyridylmethyl-N′-methylalanate;
N,N-bis(2-pyrimidylmethyl)isopropylenediamine-N′-2-pyridylmethyl-N′-methylalanate;
N,N-bis(1-1,2,4-triazolylmethyl)ethylenediamine-N′-2-pyridylmethyl-N′-acetate;
N,N-bis(1-1,2,4-triazolylmethyl)cyclohexanediamine-N′-2-pyridylmethyl-N′-acetate;
N,N-bis(1-1,2,4-triazolylmethyl)isopropylenediamine-N′-2-pyridylmethyl-N′-acetate;
N,N-bis(1-1,2,4-triazolylmethyl)ethylendiamine-N′-2-pyridylmethyl-N′-alanate;
N,N-bis(1-1,2,4-triazolylmethyl)cyclohexanediamine-N′-2-pyridylmethyl-N′-alanate;
N,N-bis(1-1,2,4-triazolylmethyl)isopropylenediamine-N′-2-pyridylmethyl-N′-alanate;
N,N-bis(1-1,2,4-triazolylmethyl)ethylendiamine-N′-2-pyridylmethyl-N′-methylalanate;
N,N-bis(1-1,2,4-triazolylmethyl)cyclohexanediamine-N′-2-pyridylmethyl-N′-methylalanate;
N,N-bis(1-1,2,4-triazolylmethyl)isopropylenediamine-N′-2-pyridylmethyl-N′-methylalanate;
N-1-pyrazolylmethyl-N,N′-bis(2-pyridylmethyl)-ethylenediamine-N′-acetate;
N-1-pyrazolylmethyl-N,N′-bis(2-pyridylmethyl)-cyclohexanediamine-N′-acetate;
N-1-pyrazolylmethyl-N,N′-bis(2-pyridylmethyl)-isopropylenediamine-N′-acetate;
N-1-pyrazolylmethyl-N,N′-bis(2-pyridylmethyl)-ethylenediamine-N′-alanate;
N-1-pyrazolylmethyl-N,N′-bis(2-pyridylmethyl)-cyclohexanediamine-N′-alanate;
N-1-pyrazolylmethyl-N,N′-bis(2-pyridylmethyl)-isopropylenediamine-N′-alanate;
N-1-pyrazolylmethyl-N,N′-bis(2-pyridylmethyl)-ethylenediamine-N′-methylalanate;
N-1-pyrazolylmethyl-N,N′-bis(2-pyridylmethyl)-cyclohexanediamine-N′-methylalanate;
N-1-pyrazolylmethyl-N,N′-bis(2-pyridylmethyl)-isopropylenediamine-N′-methylalanate;
N-2-imidazolylmethyl-N,N′-bis(2-pyridylmethyl)-ethylenediamine-N′-acetate;
N-2-imidazolylmethyl-N,N′-bis(2-pyridylmethyl)-cyclohexanediamine-N′-acetate;
N-2-imidazolylmethyl-N,N′-bis(2-pyridylmethyl)-isopropylenediamine-N′-acetate;
N-2-imidazolylmethyl-N,N′-bis(2-pyridylmethyl)-ethylenediamine-N′-alanate;
N-2-imidazolylmethyl-N,N′-bis(2-pyridylmethyl)-cyclohexanediamine-N′-alanate;
N-2-imidazolylmethyl-N,N′-bis(2-pyridylmethyl)-isopropylenediamine-N′-alanate;
N-2-imidazolylmethyl-N,N′-bis(2-pyridylmethyl)-ethylenediamine-N′-methylalanate;
N-2-imidazolylmethyl-N,N′-bis(2-pyridylmethyl)-cyclohexanediamine-N′-methylalanate;
N-2-imidazolylmethyl-N,N′-bis(2-pyridylmethyl)-isopropylenediamine-N′-methylalanate;
N-1-1,2,4-triazolylmethyl-N,N′-bis(2-pyridylmethyl)-ethylenediamine-N′-acetate;
N-1-1,2,4-triazolylmethyl-N,N′-bis(2-pyridylmethyl)-cyclohexanediamine-N′-acetate;
N-1-1,2,4-triazolylmethyl-N,N′-bis(2-pyridylmethyl)-isopropylenediamine-N′-acetate;
N-1-1,2,4-triazolylmethyl-N,N′-bis(2-pyridylmethyl)-ethylenediamine-N′-alanate;
N-1-1,2,4-triazolylmethyl-N,N′-bis(2-pyridylmethyl)-cyclohexanediamine-N′-alanate;
N-1-1,2,4-triazolylmethyl-N,N′-bis(2-pyridylmethyl)-isopropylenediamine-N′-alanate;
N-1-1,2,4-triazolylmethyl-N,N′-bis(2-pyridylmethyl)-ethylenediamine-N′-methylalanate;
N-1-1,2,4-triazolylmethyl-N,N′-bis(2-pyridylmethyl)-cyclohexanediamine-N′-methylalanate;
N-1-1,2,4-triazolylmethyl-N,N′-bis(2-pyridylmethyl)-isopropylenediamine-N′-methylalanate;
N-2-pyrimidylmethyl-N,N′-bis(2-pyridylmethyl)-ethylenediamine-N′-acetate;
N-2-pyrimidylmethyl-N,N′-bis(2-pyridylmethyl)-cyclohexanediamine-N′-acetate;
N-2-pyrimidylmethyl-N,N′-bis(2-pyridylmethyl)-isopropylenediamine-N′-acetate;
N-2-pyrimidylmethyl-N,N′-bis(2-pyridylmethyl)-ethylenediamine-N′-alanate;
N-2-pyrimidylmethyl-N,N′-bis(2-pyridylmethyl)-cyclohexanediamine-N′-alanate;
N-2-pyrimidylmethyl-N,N′-bis(2-pyridylmethyl)-isopropylenediamine-N′-alanate;
N-2-pyrimidylmethyl-N,N′-bis(2-pyridylmethyl)-ethylenediamine-N′-methylalanate;
N-2-pyrimidylmethyl-N,N′-bis(2-pyridylmethyl)-cyclohexanediamine-N′-methylalanate; or
N-2-pyrimidylmethyl-N,N′-bis(2-pyridylmethyl)-isopropylenediamine-N′-methylalanate.

Further preferred ligands include the above-listed ligands wherein the heteroaryl group(s) and/or the bridging ethylene-, cyclohexane- and isopropylene-group is, independently of the other such group(s), substituted by methyl, ethyl, methoxy, ethoxy and/or hydroxy. The most preferred substituents of the heteroaryl group(s) and the respective bridging groups are methyl and ethyl.

Particularly preferred ligands include:
N,N,N′-tris(2-pyridylmethyl)ethylenediamine-N′-acetate;
N,N,N′-tris(2-pyridylmethyl)cyclohexanediamine-N′-acetate;
N,N,N′-tris(2-pyridylmethyl)isopropylenediamine-N′-acetate;
N,N,N′-tris(1-pyrazolylmethyl)ethylenediamine-N′-acetate;
N,N,N′-tris(2-imidazolylmethyl)ethylenediamine-N′-acetate;
N,N,N′-tris(2-pyrimidylmethyl)ethylenediamine-N′-acetate; and
N,N,N′-tris(1-1,2,4-triazolylmethyl)ethylenediamine-N′-acetate,

as well as the foregoing ligands wherein the ethylene-, cyclohexane- and isopropylene-group is substituted by methyl, ethyl, methoxy, ethoxy and/or hydroxy.

Further particularly preferred ligands include:
N,N,N′-tris(2-pyridylmethyl)ethylenediamine-N′-acetate;
N,N,N′-tris(2-pyridylmethyl)cyclohexanediamine-N′-acetate; and
N,N,N′-tris(2-pyridylmethyl)isopropylenediamine-N′-acetate,

wherein the heteroaryl group(s) are substituted by methyl, ethyl, methoxy, ethoxy and/or hydroxy.

The most preferred ligand is: N,N,N′-tris(2-pyridylmethyl)ethylenediamine-N′-acetate, herein after referred to as (tpena⁻).

Preferably, the coordinating atoms/molecules X in formula (III) are selected from the group consisting of H₂O; alkyl-CN, particularly CH₃CN; Cl⁻; O²⁻; OH⁻; OO-alkyl and OOH⁻. Further preferred groups X include the oxygen-coordinating terminal oxidants of dihydrogen peroxide (i.e., coordinated in its deprotonated form, namely hydrogen peroxide); alkyl peroxides; iodosylaryl, particularly iodosylbenzene; hypochlorite, perborate, amine N-oxides such as trialkylamine N-oxides, morpholine N-oxide and pyridine N-oxide.

Suitable counter ions Y are anions which give rise to the formation of storage-stable solids. RCOO⁻, F⁻, Cl⁻, Br⁻, NO₃⁻, BPh₄⁻, ClO₄⁻, BF₄⁻, PF₆⁻, RSO₃⁻, RSO₄⁻, SO₄²⁻, CF₃SO₃⁻, PO₄³⁻, RPO₄²⁻, with R being selected from H, optionally substituted alkyl or optionally substituted aryl. A particularly preferred counter ion is ClO₄⁻.

The iron or manganese in the complexes of the invention are in the II, III, IV or V oxidation state in solution and in the II or III oxidation state in the solid state. When the iron or manganese complexes of the invention are in solution, depending on the pH of the solution, any non-coordinated heteroaryl group of the ligand L may be protonated. The pH at which such protonation occurs depends on the nature of the metal, the ligand, the coordinating species X and the solvent composition. In aqueous solution, any non-coordinated heteroaryl group is mostly protonated at a pH below 5, it being understood that the protonated and deprotonated forms are in equilibrium at all pHs.

While not wishing to be bound by a particular mechanism of action, it is postulated that the carboxylate donor permits the redox properties of the iron and manganese complexes to be tuned and, in turn, encourage the formation of metal based oxidants for involvement in the oxidation of various substrates. The hexadentate ligand system of the complexes of the present invention contains an inherent flexibility for alternative decoordination of either a heteroaryl or carboxylate group during reactions, which is believed to be useful for supporting various oxidation states and geometries. As a consequence of being able to support the inherent changes in coordination number which may be mechanistically required for the catalysts, the present hexadentate ligand system is more coordinatively flexible than the ligands employed in the state of the art, and thereby provides the iron and manganese complexes of the present invention with more effective catalytic activity.

In accordance with a further aspect of the present invention, a process is provided for the preparation of reactive complex-oxidant adducts of formula (III) as described herein above, wherein the coordinating species X is an oxidant selected from the group consisting of peroxides, peracids, iodosylaryls, hypohalides, oxone, N-oxides and dioxygen.

The process for the preparation of [LM(X)]^{z}Y_{q} according to formula (III) as defined above, wherein the coordinating species X is an oxidant selected from the group consisting of peroxides, peracids, iodosylaryls, hypohalides, oxone, N-oxides and dioxygen, comprises the step of:
mixing [(LH)₂Fe₂(O)]^{z}Y_{q} according to formula (I) as defined above or [LM]^{z}Y_{q} according to formula (II) as defined above with an oxidant in an organic solvent, such as acetonitrile, wherein the oxidant is selected from the group consisting of peroxides, peracids, iodosylaryls, hypohalides, oxone, N-oxides and dioxygen.

The process may further comprise the step of precipitating [LM(X)]^{z}Y_{q} from the solution by the addition of an appropriate solvent, such as diethylether.

Examples of suitable oxidants include dihydrogen peroxide, alkyl peroxides, iodosylaryl and, in particular, iodosylbenzene, hypochlorite, perborate, oxone, amine N-oxides such as trialkyl N-oxides, morpholine N-oxide, 4-methylmorpholine N-oxide, pyridine N-oxide, and dioxygen.

The above-described process is particularly undertaken for the preparation of [LM(X)]^{z}Y_{q} according to formula (III), wherein M is Fe; L is N,N,N′-tris(2-pyridylmethyl)ethylendiamine-N′-acetate; X is selected from the group consisting of iodosylbenzene, alkyl-OO⁻, HOO⁻, and O²⁻; and Y is ClO₄⁻.

Iodosylaryls such as iodosylbenzene are an important class of oxygen atom transfer reagents in organic synthesis, often used in conjunction with transition metal-based catalysts. However, iodosylbenzene in its unsubstituted form is very insoluble. Notably, the coordination of iodosylbenzene in iron or manganese complexes of the present invention facilitates its dissolution by direct reaction, thereby rendering it more useful for certain oxidation reactions.

The iron and manganese complexes of the present invention having general formulas (I), (II) and (III) as described herein above provide novel catalysts for oxidation reactions. Depending on the nature of the chemical oxidant employed in the oxidation reactions, the iron or manganese complexes may be the catalytically competent species during catalysis or may be the direct precursor of a catalytically competent species. As already indicated herein above, the iron or manganese ion is in the II, III, IV or V oxidation state in solution.

As noted herein above in relation to the process for the preparation of reactive complex-oxidant adducts of formula (III), iron and manganese complexes of the present invention can bind oxidants directly. Examples of such oxidants include those commonly employed for the oxidation of organic substrates such as sulfides; alkenes, including aromatics; amines; alkanes; and alcohols, as exemplified by dihydrogen peroxide, alkyl peroxides, iodosylaryls, hypochlorite, perborate, oxone, amine N-oxides and dioxygen.

The iron and manganese complexes of the present invention may be employed for the catalysis of the oxidation of organic substrates. The manganese complexes of the present invention can also be successfully employed for the catalysis of the oxidation of water.

In aqueous solution, active catalysts are provided and/or generated by the iron and manganese complexes of the present invention at pHs between about 2-8.

The use of the iron and manganese complexes of the present invention as oxidation (pro)-catalysts is illustrated schematically below in relation to the oxidation of a number of typical organic substrates using a terminal oxidant generalized by “DO” (e.g., D=C₆H₅I, R₃N, RO, H₂O, Cl, B, O). The catalytic potential of the iron and manganese complexes of formulas (I), (II) and (III) is not, however, limited to reactions of this type and, as already indicated above, the manganese complexes of the present invention also catalyze the oxidation of water.

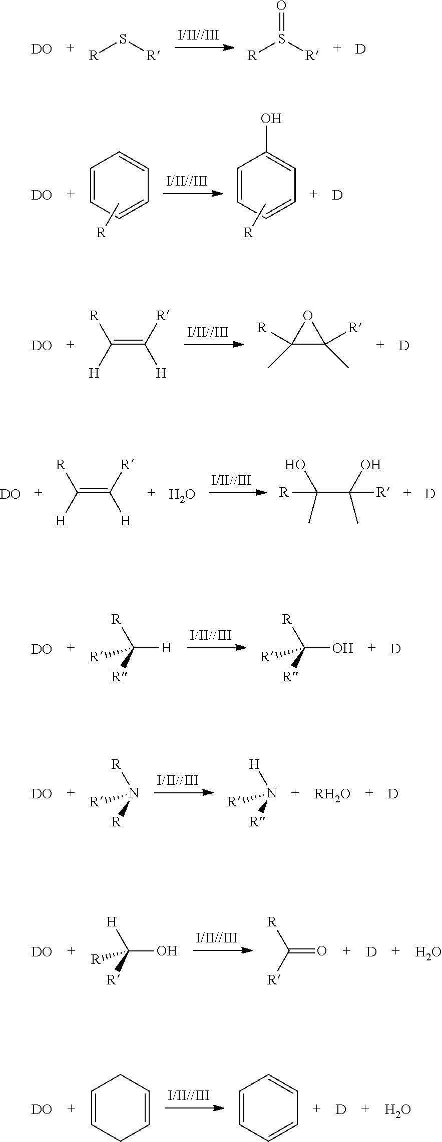

In accordance with a further aspect of the present invention, a method is provided for the catalysis of oxidation reactions, which method comprises combining an iron or manganese complex of the invention having formula (I), (II) or (III) with the compound to be oxidized in solution, together with an appropriate oxidant and, after completion of the oxidation reaction, isolating the oxidized product.

The foregoing method of the invention may be employed for the catalysis of the oxidation of organic substrates. Such method comprises combining an iron or manganese complex of the invention having formula (I), (II) or (III) with the organic substrate which is to be oxidized in an organic solvent, together with an appropriate oxidant and, after completion of the oxidation reaction, isolating the oxidized product.

The reaction conditions, including the nature of solvents and any possible co-solvents, employed in these methods will vary depending on both the nature of the organic substrate to be oxidized and the nature of the oxidant. The reaction conditions to be implemented for the catalytic oxidation of typical organic substrates are well known in the art. See, for example, “Transition metals in organic synthesis—a practical approach”, Ed. Susan E. Gibson, 1997 Oxford University Press; V. Conte, A. Coletti, B. Floris, G. Licini, C. Zonta, COORDINATION CHEMISTRY REVIEWS, (2011) 255, 2165-2177; and C. L. Sun, B. J. Li, Z. J. Shi, 2011 Chemistry Reviews (2011) 1293-1314, the disclosures of all of which are incorporated herein by reference.

The foregoing method of the invention may also be employed for the catalysis of the oxidation of water. Such method comprises adding an appropriate oxidant to an aqueous solution of a manganese complex of the invention having formula (II) or (III), which aqueous solution may optionally further include an organic solvent, and isolating the oxidized product upon completion of the oxidation reaction.

Suitable oxidants include, e.g., tert-butyl hydrogen peroxide, (NH₄)₂[Ce(NO₃)₆], Ce(ClO₄)₄, oxone, and photo-oxidation mediated by [Ru(bipyridine)₃]²⁺ in the presence of an electron acceptor. Again, the reaction conditions, including the nature of solvents and any possible co-solvents, employed in these methods will vary depending on the nature of the oxidant employed. The reaction conditions to be implemented are well known in the art, as illustrated in the above-indicated publications.

The invention will now be further illustrated by way of the following non-limiting examples.

### EXAMPLES

### Synthesis of Representative Pro-Catalysts

### Example 1

### Synthesis of N,N,N′-tris(2-pyridylmethyl)ethylendiamine-N′-acetic acid: tpenaH

A mixture of N,N,N′-tris(2-pyridylmethyl)ethylendiamine (0.1212 g, 0.36 mmol), bromoacetic acid (0.0513 g, 0.37 mmol) and Cs₂CO₃ (0.2208 g, 1.14 mmol) in absolute ethanol (4 ml) was heated overnight under reflux and a nitrogen atmosphere. The solvent was removed under reduced pressure and the residue was redissolved in chloroform to precipitate CsBr which was filtered off. The filtrate was evaporated in vacuo leaving a brown oil (0.15 g). δ_{c} (CDCl₃): 51.774, 51.795 (NCH₂CH₂N), 60.056, 60.401, 60.723, 60.820 (2×NCH₂C₅H₄N, NC′H₂C₅H₄N, NCH₂COOH), 121.940, 121.999, 122.077, 122.240, 123.130, 123.666, 124.137 (2×C³ py, 2×C⁵ py, C³′ py, C⁵′ py), 136.379, 136.409, 136.532 (2×C⁴ py, C⁴′ py), 149.134, 149.187, 149.420 (2×C⁶ py, C⁶′ py), 158.899, 159.380 (2×C² py, C²′ py), 176.042 (C═O).

### Synthesis of Complex of Formula I: [(tpenaH)Fe(μ-O)Fe(tpenaH)](ClO₄)₄(H₂O)₂

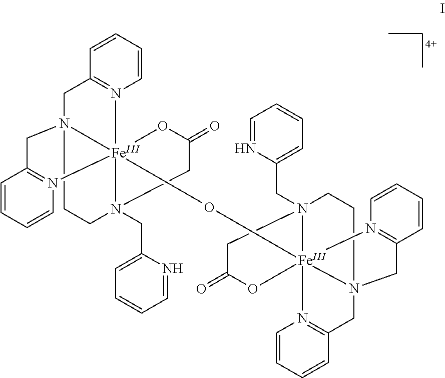

A solution of Fe(ClO₄)₃.xH₂O (0.25 mmol) in water (1.5 ml) was added to a solution of N,N,N′-tris(2-pyridylmethyl)ethylendiamine-N′-acetato (tpenaH) (0.25 mmol) and 70% perchloric acid (3 drops) in water (1.5 ml). The mixture was left standing under ambient conditions upon which the compound precipitates. The product is washed with a few drops of water and dried by suction. Yield: 80%. IR (KBr): 3434 (br), 3077 (w), 1662 (s), 1609 (s), 1572 (w), 1467 (m), 1446 (m), 1352 (m), 1303 (m), 1121 (s), 1025 (m), 955 (w), 924 (w), 837 (s), 771 (m), 625 (s), 495 (w) cm⁻¹. Anal. calc. for FeC₄₄H₅₀Cl₄N₁₀O₂₃: C, 39.60; H, 3.75; N, 10.42. Found: C, 39.71; H, 3.77; N, 10.49.

### Example 2

### Synthesis of Complex of Formula II: [M(tpena)]⁺ where M=Mn(II) or Fe(III)

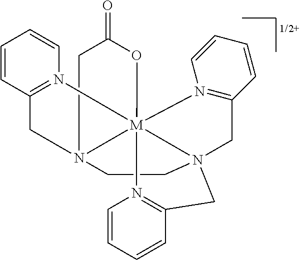

[(tpenaH)Fe(μ-O)Fe(tpenaH)](ClO₄)₄(H₂O)₂ prepared as in Example 1 or [(tpena)Mn(OH₂)](ClO₄) prepared as in Example 5 below is dissolved in an organic solvent such as acetonitrile. This affects the respective dehydrations of the complexes to give the solution species [Fe(tpena)]²⁺ or [Mn(tpena)]⁺.

### Example 3

### Synthesis of Complex of Formula III: [Fe(tpena)OO^{t}Bu]⁺ or [Fe(tpena)OOH]⁺

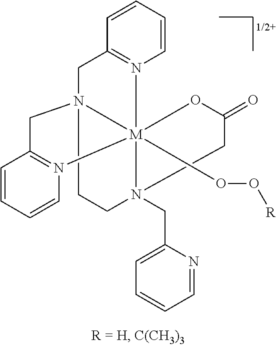

[(tpenaH)Fe(μ-O)Fe(tpenaH)](ClO₄)₄(H₂O)₂ prepared as in Example 1 is dissolved in an organic solvent such as acetonitrile or methanol and one or more equivalents of tert-butylhydrogen peroxide or dihydrogen peroxide is added. The initially yellow solution turns purple due to the formation of the peroxo-complex. The half-life of these reactive species in solution is seconds to hours.

EPR spectrum (MeCN, 10K) [Fe(OOH)(tpena)]⁺, g=2.13, 2.13, 1.98. UV-vis λₘₐₓ=514 nm.

EPR spectrum (MeCN, 100K) [Fe(OOC(CH₃)₃)(tpena)]⁺, g-values at (2.20, 2.13, 1.97).

Not detectable by ESI MS.

### Example 4

### Synthesis of Complex of Formula III: [Fe(tpena)OIPh](ClO₄)₂

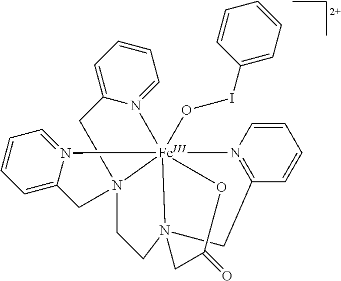

[(tpenaH)Fe(μ-O)Fe(tpenaH)](ClO₄)₄(H₂O)₂ (45 mg, 0.050 mmol) prepared as in Example 1 was dissolved in acetonitrile (4 ml) and iodosylbenzene (22 mg, 0.10 mmol) was added. The mixture was stirred for 30 mins. until a clear red solution was obtained. Diethyl ether (8 ml) was added and an orange powder precipitated. This was collected and dried in a stream of nitrogen to give [Fe(tpena)OIPh](ClO₄)₂(MeCN)(H₂O)_{0.5} as an orange-red powder. Yield: 86 mg (94%). Anal. calc. for Fe₂C₅₈H₆₃I₂Cl₄N₁₁O₂₃: C, 38.91; H, 3.55; N, 8.61. Found: C, 38.82; H, 3.37; N, 8.87. IR: 3552 (br), 3084 (m), 1600 (s), 1571 (m), 1470 (m), 1440 (m), 1389 (m), 1297 (w), 1192 (w), 1067 (s), 993 (m), 947 (m), 865 (w), 834 8w), 765 (m), 743 (m), 679 (m), 650 8m), 619 (s), 529 (w), 490 (w) cm⁻¹. UV-Vis (CH₃CN): 422 nm, (sh, 1100 mol⁻¹dm³cm⁻¹). ESIMS (CH₃CN): m/z 765.0071 (15%, [Fe^{III}(tpena)(OIPh)(ClO₄)]⁺.

### Example 5

### Synthesis of Complex of Formula III: [Mn(tpena)OH₂](ClO₄)

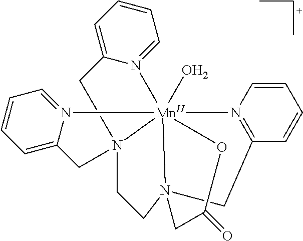

Mn(ClO₄)₂.6H₂O (1.4 mmol) and N,N,N′-tris(2-pyridylmethyl)ethylendiamine-N′-acetato (tpenaH) (1.3 mmol) prepared as in Example 1 were dissolved in a mixture of methanol (6 ml) and water (5 ml). Slow evaporation of the solvents afforded the product [(tpena)Mn(OH₂)](ClO₄) as a beige microcrystalline precipitate. Yield: 70%. ESI MS (CH₃CN) m/z: 445.1 ([Mn(tpena)]⁺, 100%), 989.2 ({[Mn₂(tpena-H)(tpena)]ClO₄}⁺, 50%). IR (KBr) v (cm⁻¹): 1605 (C═O, s), 1092 (ClO₄⁻, vs). Anal. calc. (%) for C₂₃H₃₆N₅O₁₁ClMn ([Mn(L⁴)(OH₂)]ClO₄.3H₂O.CH₃OH)C, 42.57; H, 5.59 N, 10.79. Found C, 42.49; H, 4.49; N, 10.85.

### Example 6

### Synthesis of [Fe^{IV}(O)(tpenaH)]²⁺

[(tpenaH)Fe(μ-O)Fe(tpenaH)](ClO₄)₄(H₂O)₂ prepared as in Example 1 is dissolved in water and one or more equivalents of cerium ammonium nitrate is added. The solution turns green due to the formation of the iron (IV) oxo-complex, [Fe^{IV}(O)(tpenaH)]²⁺. UV-vis λₘₐₓ=730 Mössbauer spectroscopy isomer shift and quadrupole splitting of δ=0.00 mms⁻¹ and ΔEQ=0.90 mms⁻¹.

### Catalytic Oxidation of Thioanisol

### Example 7

Thioanisol (310 μl, 2.5 mmol) and [(tpenaH)Fe(μ-O)Fe(tpenaH)](ClO₄)₄(H₂O)₂ (1.65 mg, 1.2 μmol) prepared as in Example 1 were dissolved in acetonitrile (3 ml). Iodosylbenzene, PhIO, (550 mg, 2.5 mmol) was added and the mixture was stirred under nitrogen at 50° C. After 2 h, a clear solution was obtained. The solution was evaporated, the residue was extracted with CDCl₃, filtered through a celite plug and analyzed by ¹H NMR spectroscopy. When the experiment was repeated in the absence of catalyst, only thioanisol was detected by ¹H NMR.

In another experiment, second equivalent portions of thioanisol and PhIO were added after the complete disappearance of the suspension of iodosylbenzene from the first addition. A similar workup was carried out after dissolution of this second portion of iodosylbenzene (1.5 hours). This showed twice the yield to that obtained above.

### Catalytic Oxidation of Alkenes

### Example 8

### Epoxidation of Trans-Stilbene to Give Trans-Stilbene Oxide

Trans-stilbene (215 mg, 1.19 mmol) and 4-methylmorpholine-N-oxide (139 mg, 1.187 mmol) were dissolved in 4 ml MeCN and the solution is heated to 50° C. A solution of [(tpenaH)Fe(μ-O)Fe(tpenaH)](ClO₄)₄(H₂O)₂ (24 mg, 0.0179 mmol) in 4 ml MeCN was added with a syringe through a septum. The reaction is left for 3 days at 50° C. 10 ml of water was added and the mixture was washed with 2×10 ml of diethylether. The ether phase was washed with 10 ml water, separated and evaporated under vacuum to give the product. Yield 80.3%. No signal for possible cis-stilbene oxide, 4-methylmorpholine-N-oxide or benzaldehyde are present in the product (verified by NMR).

### Catalytic Oxidation of Water

### Example 9

Cold tert-butyl hydrogen peroxide (TBHP) (6.4 M in water, X ml, X ranging from 0.25 to 2.0) was added to a test tube containing an aqueous solution of [Mn(tpena)OH₂](ClO₄) (1 mL, 0.2 mM in [Mn]) as prepared in Example 5 and acetonitrile (4-X ml) immersed in a water bath at 40° C. (controlled using a Lauda ecoline RE104 thermostat). In a second series of experiments, the concentration of [Mn(tpena)OH₂](ClO₄) was varied while the TBHP concentration was kept constant. Here acetonitrile (1 ml) and then cold TBHP (6.4 M in water, 1 ml) was added to an aqueous solution (1 ml) of [Mn(mcbpen)]ₙ(ClO4)ₙ.nH₂O (0.050 to 0.50 mmol in nMn; mcbpen=N-methyl-N′-carboxymethyl-N,N′-bis(2-pyridylmethyl)ethane-1,2-diamine). The test tube formed part of a closed system and the gas that was evolved was led through a reflux condenser and was collected in a low friction, air tight glass syringe at room temperature. The amount of oxygen evolved was calculated by volume at ambient air pressure and temperature. Over 90% yields recorded over 10,000 TONs recorded [mol of O₂ per mol of [Mn(tpena)OH₂](ClO₄)].

The disclosures of each publication, including patents, patent applications, and scientific literature, cited or described herein are incorporated herein by reference, in their entireties.

The present invention is not to be limited in scope by the specific embodiments described herein. Indeed, various modifications of the invention in addition to those described herein will be apparent to those skilled in the art from the foregoing description. Such modifications are intended to fall within the scope of the appended claims.

I claim:

1. An iron or manganese complex having general formula (I), (II) or (III):
[(LH)₂Fe₂(O)]^{z}Y_{q} or [(L)₂Fe₂(O)]^{z}Y_{q} (I)
[LM]^{z}Y_{q} (II)
[LM(X)]^{z}Y_{q} (III)
wherein:
M represents a metal ion selected from Fe or Mn in the II, III, IV or V oxidation state;
L represents a monoanionic potentially hexadentate ligand containing one carboxylate group and five nitrogen donor atoms, wherein the ligand coordinates to the Fe or Mn metal ion in the complex via said carboxylate group and at least four of said nitrogen donor atoms and has general formula (IV):
R¹R¹N-A-N(R¹)WC(O)O⁻ (IV)
wherein:
each R¹ independently represents —R²—B, in which R² represents an optionally substituted group selected from the group consisting of alkylene, alkenylene, oxyalkylene, aminoalkylene and alkylene ether, and B represents an optionally substituted heteroaryl group selected from the group consisting of pyridinyl, pyrazinyl, pyrazolyl, imidazolyl, benzimidazolyl, pyrimidinyl, triazolyl and thiazolyl;
A represents an optionally substituted alkylene or arylene bridging group selected from the group consisting of —CH₂CH₂—, —CH₂CH₂CH₂—, —CH₁₀—, and —C₆H₄; and
W represents a group selected from the group consisting of alkylene, arylene and arylalkylene;
X represents a coordinating species selected from the group consisting of oxygen-coordinating terminal oxidants including peroxides, peracids, iodosylaryls, hypohalides, oxone, N-oxides and dioxygen; H₂O, ROH, RCN, OH⁻, RO⁻, RCOO⁻, F⁻, Cl⁻, Br⁻, I⁻, O²⁻, NO₃⁻, NO₂⁻, ClO₄⁻; and aromatic nitrogen-donors selected from the group consisting of pyridines, pyrazines, pyrazoles, imidazoles, benzimidazoles, pyrimidines, triazoles and thiazoles, with R being selected from H, optionally substituted alkyl or optionally substituted aryl;
Y represents a non-coordinating counter ion;
z represents the charge of the complex and is an integer which can be positive or zero; and
q=z/[charge Y].

2. The iron or manganese complex according to claim 1, wherein L is selected from:
N,N,N′-tris(2-pyridylmethyl)ethylenediamine-N′-acetate;
N,N,N′-tris(2-pyridylmethyl)cyclohexanediamine-N′-acetate;
N,N,N′-tris(2-pyridylmethyl)isopropylenediamine-N′-acetate;
N,N,N′-tris(2-pyridylmethyl)ethylenediamine-N′-alanate;
N,N,N′-tris(2-pyridylmethyl)cyclohexanediamine-N′-alanate;
N,N,N′-tris(2-pyridylmethyl)isopropylenediamine-N′-alanate;
N,N,N′-tris(2-pyridylmethyl)ethylenediamine-N′-methylalanate;
N,N,N′-tris(2-pyridylmethyl)cyclohexanediamine-N′-methylalanate;
N,N,N′-tris(2-pyridylmethyl)isopropylenediamine-N′-methylalanate;
N,N,N′-tris(1-pyrazolylmethyl)ethylenediamine-N′-acetate;
N,N,N′-tris(1-pyrazolylmethyl)cyclohexanediamine-N′-acetate;
N,N,N′-tris(1-pyrazolylmethyl)isopropylenediamine-N′-acetate;
N,N,N′-tris(1-pyrazolylmethyl)ethylenediamine-N′-alanate;
N,N,N′-tris(1-pyrazolylmethyl)cyclohexanediamine-N′-alanate;
N,N,N′-tris(1-pyrazolylmethyl)isopropylenediamine-N′-alanate;
N,N,N′-tris(1-pyrazolylmethyl)ethylenediamine-N′-methylalanate;
N,N,N′-tris(1-pyrazolylmethyl)cyclohexanediamine-N′-methylalanate;
N,N,N′-tris(1-pyrazolylmethyl)isopropylenediamine-N′-methylalanate;
N,N,N′-tris(2-imidazolylmethyl)ethylenediamine-N′-acetate;
N,N,N′-tris(2-imidazolylmethyl)cyclohexanediamine-N′-acetate;
N,N,N′-tris(2-imidazolylmethyl)isopropylenediamine-N′-acetate;
N,N,N′-tris(2-imidazolylmethyl)ethylenediamine-N′-alanate;
N,N,N′-tris(2-imidazolylmethyl)cyclohexanediamine-N′-alanate;
N,N,N′-tris(2-imidazolylmethyl)isopropylenediamine-N′-alanate;
N,N,N′-tris(2-imidazolylmethyl)ethylenediamine-N′-methylalanate;
N,N,N′-tris(2-imidazolylmethyl)cyclohexanediamine-N′-methylalanate;
N,N,N′-tris(2-imidazolylmethyl)isopropylenediamine-N′-methylalanate;
N,N,N′-tris(2-pyrimidylmethyl)ethylenediamine-N′-acetate;
N,N,N′-tris(2-pyrimidylmethyl)ethylenediamine-N′-acetate;
N,N,N′-tris(2-pyrimidylmethyl)cyclohexanediamine-N′-acetate;
N,N,N′-tris(2-pyrimidylmethyl)isopropylenediamine-N′-acetate;
N,N,N′-tris(2-pyrimidylmethyl)ethylenediamine-N′-alanate;
N,N,N′-tris(2-pyrimidylmethyl)cyclohexanediamine-N′-alanate;
N,N,N′-tris(2-pyrimidylmethyl)isopropylenediamine-N′-alanate;
N,N,N′-tris(2-pyrimidylmethyl)ethylenediamine-N′-methylalanate;
N,N,N′-tris(2-pyrimidylmethyl)cyclohexanediamine-N′-methylalanate;
N,N,N′-tris(2-pyrimidylmethyl)isopropylenediamine-N′-methylalanate;
N,N,N′-tris(1-1,2,4-triazolylmethyl)ethylenediamine-N′-acetate;
N,N,N′-tris(1-1,2,4-triazolylmethyl)cyclohexanediamine-N′-acetate;
N,N,N′-tris(1-1,2,4-triazolylmethyl)isopropylenediamine-N′-acetate;
N,N,N′-tris(1-1,2,4-triazolylmethyl)ethylenediamine-N′-alanate;
N,N,N′-tris(1-1,2,4-triazolylmethyl)cyclohexanediamine-N′-alanate;
N,N,N′-tris(1-1,2,4-triazolylmethyl)isopropylenediamine-N′-alanate;
N,N,N′-tris(1-1,2,4-triazolylmethyl)ethylenediamine-N′-methylalanate;
N,N,N′-tris(1-1,2,4-triazolylmethyl)cyclohexanediamine-N′-methylalanate;
N,N,N′-tris(1-1,2,4-triazolylmethyl)isopropylenediamine-N′-methylalanate;
N,N-bis(2-pyridylmethyl)ethylenediamine-N′-1-pyrazolylmethyl-N′-acetate;
N,N-bis(2-pyridylmethyl)cyclohexanediamine-N′-1-pyrazolylmethyl-N′-acetate;
N,N-bis(2-pyridylmethyl)isopropylenediamine-N′-1-pyrazolylmethyl-N′-acetate;
N,N-bis(2-pyridylmethyl)ethylenediamine-N′-1-pyrazolylmethyl-N′-alanate;
N,N-bis(2-pyridylmethyl)isopropylenediamine-N′-1-pyrazolylmethyl-N′-alanate;
N,N-bis(2-pyridylmethyl)isopropylenediamine-N′1-pyrazolylmethyl-N′-alanate;
N,N-bis(2-pyridylmethyl)ethylenediamine-N′-1-pyrazolylmethyl-N′-methylalanate;
N,N-bis(2-pyridylmethyl)isopropylenediamine-N′1-pyrazolylmethyl-N′-methylalanate;
N,N-bis(2-pyridylmethyl)isopropylenediamine-N′-1-pyrazolylmethyl-N′-methylalanate;
N,N-bis(2-pyridylmethyl)ethylenediamine-N′-2-imidazolylmethyl-N′-acetate;
N,N-bis(2-pyridylmethyl)cyclohexanediamine-N′2-imidazolylmethyl-N′-acetate;
N,N-bis(2-pyridylmethyl)isopropylenediamine-N′-2-imidazolylmethyl-N′-acetate;
N,N-bis(2-pyridylmethyl)ethylenediamine-N′-1-2-imidazolylmethyl-N′-alanate;
N,N-bis(2-pyridylmethyl)cyclohexanediamine-N′-1-2-imidazolylmethyl-N′-alanate;
N,N-bis(2-pyridylmethyl)isopropylenediamine-N′-1-2-imidazolylmethyl-N′-alanate;
N,N-bis(2-pyridylmethyl)ethylenediamine-N′-1-2-imidazolylmethyl-N′-methylalanate;
N,N-bis(2-pyridylmethyl)cyclohexanediamine-N′-1-2-imidazolylmethyl-N′-methylalanate;
N,N-bis(2-pyridylmethyl)isopropylenediamine-N′-1-2-imidazolylmethyl-N′-methylalanate;
N,N-bis(2-pyridylmethyl)ethylenediamine-N′-2-pyrimidylmethyl-N′-acetate;
N,N-bis(2-pyridylmethyl)cyclohexanediamine-N′-2-pyrimidylmethyl-N′-acetate;
N,N-bis(2-pyridylmethyl)isopropylenediamine-N′-2-pyrimidylmethyl-N′-acetate;
N,N-bis(2-pyridylmethyl)ethylenediamine-N′-2-pyrimidylmethyl-N′-alanate;
N,N-bis(2-pyridylmethyl)cyclohexanediamine-N′-2-pyrimidylmethyl-N′-alanate;
N,N-bis(2-pyridylmethyl)isopropylenediamine-N′-2-pyrimidylmethyl-N′-alanate;
N,N-bis(2-pyridylmethyl)ethylenediamine-N′-2-pyrimidylmethyl-N′-methylalanate;
N,N-bis(2-pyridylmethyl)cyclohexanediamine-N′-2-pyrimidylmethyl-N′-methylalanate;
N,N-bis(2-pyridylmethyl)isopropylenediamine-N′-2-pyrimidylmethyl-N′-methylalanate;
N,N-bis(2-pyridylmethyl)ethylenediamine-N′-1-1,2,4-triazolylmethyl-N′-acetate;
N,N-bis(2-pyridylmethyl)cyclohexanediamine-N′-1,2,4-triazolylmethyl-N′-acetate;
N,N-bis(2-pyridylmethyl)isopropylenediamine-N′-1,2,4-triazolylmethyl-N′-acetate;
N,N-bis(2-pyridylmethyl)ethylenediamine-N′-1,2,4-triazolylmethyl-N′-alanate;
N,N-bis(2-pyridylmethyl)cyclohexanediamine-N′-1,2,4-triazolylmethyl-N′-alanate;
N,N-bis(2-pyridylmethyl)isopropylenediamine-N′-1,2,4-triazolylmethyl-N′-alanate;
N,N-bis(2-pyridylmethyl)ethylenediamine-N′-1,2,4-triazolylmethyl-N′-methylalanate;
N,N-bis(2-pyridylmethyl)cyclohexanediamine-N′-1,2,4-triazolylmethyl-N′-methylalanate;
N,N-bis(2-pyridylmethyl)isopropylenediamine-N′-1,2,4-triazolylmethyl-N′—N,N-bis(1-pyrazolylmethyl)ethylenediamine-N′-2-pyridylmethyl-N′-acetate;
N,N-bis(1-pyrazolylmethyl)cyclohexanediamine-N′-2-pyridylmethyl-N′-acetate;
N,N-bis(1-pyrazolylmethyl)isopropylenediamine-N′-2-pyridylmethyl-N′-acetate;
N,N-bis(1-pyrazolylmethyl)ethylendiamine-N′-2-pyridylmethyl-N′-alanate;
N,N-bis(1-pyrazolylmethyl)cyclohexanediamine-N′-2-pyridylmethyl-N′-alanate;
N,N-bis(1-pyrazolylmethyl)isopropylenediamine-N′-2-pyridylmethyl-N′-alanate;
N,N-bis(1-pyrazolylmethyl)ethylendiamine-N′-2-pyridylmethyl-N′-methylalanate;
N,N-bis(1-pyrazolylmethyl)cyclohexanediamine-N′-2-pyridylmethyl-N′-methylalanate;
N,N-bis(1-pyrazolylmethyl)isopropylenediamine-N′-2-pyridylmethyl-N′-methylalanate;
N,N-bis(2-imidazolylmethyl)ethylenediamine-N′-2-pyridylmethyl-N′-acetate;
N,N-bis(2-imidazolylmethyl)cyclohexanediamine-N′-2-pyridylmethyl-N′-acetate;
N,N-bis(2-imidazolylmethyl)isopropylenediamine-N′-2-pyridylmethyl-N′-acetate;
N,N-bis(2-imidazolylmethyl)ethylendiamine-N′-2-pyridylmethyl-N′-alanate;
N,N-bis(2-imidazolylmethyl)cyclohexanediamine-N′-2-pyridylmethyl-N′-alanate;
N,N-bis(2-imidazolylmethyl)isopropylenediamine-N′-2-pyridylmethyl-N′-alanate;
N,N-bis(2-imidazolylmethyl)ethylendiamine-N′-2-pyridylmethyl-N′methylalanate;
N,N-bis(2-imidazolylmethyl)cyclohexanediamine-N′-2-pyridylmethyl-N′-methylalanate;
N,N-bis(2-imidazolylmethyl)isopropylenediamine-N′-2-pyridylmethyl-N′-methylalanate;
N,N-bis(2-pyrimidylmethyl)ethylenediamine-N′-2-pyridylmethyl-N′-acetate;
N,N-bis(2-pyrimidylmethyl)cyclohexanediamine-N′-2-pyridylmethyl-N′-acetate;
N,N-bis(2-pyrimidylmethyl)isopropylenediamine-N′-2-pyridylmethyl-N′-acetate;
N,N-bis(2-pyrimidylmethyl)ethylendiamine-N′-2-pyridylmethyl-N′-alanate;
N,N-bis(2-pyrimidylmethyl)cyclohexanediamine-N′-2-pyridylmethyl-N′-alanate;
N,N-bis(2-pyrimidylmethyl)isopropylenediamine-N′-2-pyridylmethyl-N′-alanate;
N,N-bis(2-pyrimidylmethyl)ethylendiamine-N′2-pyridylmethyl-N′-methylalanate;
N,N-bis(2-pyrimidylmethyl)cyclohexanediamine-N′2-pyridylmethyl-N′-methylalanate;
N,N-bis(2-pyrimidylmethyl)isopropylenediamine-N′-2-pyridylmethyl-N′-methylalanate;
N,N-bis(1-1,2,4-triazolylmethyl)ethylenediamine-N′-2-pyridylmethyl-N′-acetate;
N,N-bis(1-1,2,4-triazolylmethyl)cyclohexanediamine-N′-2-pyridylmethyl-N′-acetate;
N,N-bis(1-1,2,4-triazolylmethyl)isopropylenediamine-N′-2-pyridylmethyl-N′-acetate;
N,N-bis(1-1,2,4-triazolylmethyl)ethylendiamine-N′-2-pyridylmethyl-N′-alanate;
N,N-bis(1-1,2,4-triazolylmethyl)cyclohexanediamine-N′-2-pyridylmethyl-N′-alanate;
N,N-bis(1-1,2,4-triazolylmethyl)isopropylenediamine-N′-2-pyridylmethyl-N′-alanate;
N,N-bis(1-1,2,4-triazolylmethyl)ethylendiamine-N′-2-pyridylmethyl-N′-methylalanate;
N,N-bis(1-1,2,4-triazolylmethyl)cyclohexanediamine-N′-2-pyridylmethyl-N′-methylalanate;
N,N-bis(1-1,2,4-triazolylmethyl)isopropylenediamine-N′-2-pyridylmethyl-N′-methylalanate;
N-1-pyrazolylmethyl-N,N′-bis(2-pyridylmethyl)-ethylenediamine-N′-acetate;
N-1-pyrazolylmethyl-N,N′-bis(2-pyridylmethyl)-cyclohexanediamine-N′-acetate;
N-1-pyrazolylmethyl-N,N′-bis(2-pyridylmethyl)-isopropylenediamine-N′-acetate;
N-1-pyrazolylmethyl-N,N′-bis(2-pyridylmethyl)-ethylenediamine-N′-alanate;
N-1-pyrazolylmethyl-N,N′-bis(2-pyridylmethyl)-cyclohexanediamine-N′-alanate;
N-1-pyrazolylmethyl-N,N′-bis(2-pyridylmethyl)-isopropylenediamine-N′-alanate;
N-1-pyrazolylmethyl-N,N′-bis(2-pyridylmethyl)-ethylenediamine-N′-methylalanate;
N-1-pyrazolylmethyl-N,N′-bis(2-pyridylmethyl)-cyclohexanediamine-N′-methylalanate;
N-1-pyrazolylmethyl-N,N′-bis(2-pyridylmethyl)-isopropylenediamine-N′-methylalanate;
N-2-imidazolylmethyl-N,N′-bis(2-pyridylmethyl)-ethylenediamine-N′-acetate;
N-2-imidazolylmethyl-N,N′-bis(2-pyridylmethyl)-cyclohexanediamine-N′-acetate;
N-2-imidazolylmethyl-N,N′-bis(2-pyridylmethyl)-isopropylenediamine-N′-acetate;
N-2-imidazolylmethyl-N,N′-bis(2-pyridylmethyl)-ethylenediamine-N′-alanate;
N-2-imidazolylmethyl-N,N′-bis(2-pyridylmethyl)-cyclohexanediamine-N′-alanate;
N-2-imidazolylmethyl-N,N′-bis(2-pyridylmethyl)-isopropylenediamine-N′-alanate;
N-2-imidazolylmethyl-N,N′-bis(2-pyridylmethyl)-ethylenediamine-N′-methylalanate;
N-2-imidazolylmethyl-N,N′-bis(2-pyridylmethyl)-cyclohexanediamine-N′-methylalanate;
N-2-imidazolylmethyl-N,N′-bis(2-pyridylmethyl)-isopropylenediamine-N′-methylalanate;
N-1-1,2,4-triazolylmethyl-N,N′-bis(2-pyridylmethyl)-ethylenediamine-N′-acetate;
N-1-1,2,4-triazolylmethyl-N,N′-bis(2-pyridylmethyl)-cyclohexanediamine-N′-acetate;
N-1-1,2,4-triazolylmethyl-N,N′-bis(2-pyridylmethyl)-isopropylenediamine-N′-acetate;
N-1-1,2,4-triazolylmethyl-N,N′-bis(2-pyridylmethyl)-ethylenediamine-N′-alanate;
N-1-1,2,4-triazolylmethyl-N,N′-bis(2-pyridylmethyl)-cyclohexanediamine-N′-alanate;
N-1-1,2,4-triazolylmethyl-N,N′-bis(2-pyridylmethyl)-isopropylenediamine-N′-alanate;
N-1-1,2,4-triazolylmethyl-N,N′-bis(2-pyridylmethyl)-ethylenediamine-N′-methylalanate;
N-1-1,2,4-triazolylmethyl-N,N′-bis(2-pyridylmethyl)-cyclohexanediamine-N′-methylalanate;
N-1-1,2,4-triazolylmethyl-N,N′-bis(2-pyridylmethyl)-isopropylenediamine-N′-methylalanate;
N-2-pyrimidylmethyl-N,N′-bis(2-pyridylmethyl)-ethylenediamine-N′-acetate;
N-2-pyrimidylmethyl-N,N′-bis(2-pyridylmethyl)-cyclohexanediamine-N′-acetate;
N-2-pyrimidylmethyl-N,N′-bis(2-pyridylmethyl)-isopropylenediamine-N′-acetate;
N-2-pyrimidylmethyl-N,N′-bis(2-pyridylmethyl)-ethylenediamine-N′-alanate;
N-2-pyrimidylmethyl-N,N′-bis(2-pyridylmethyl)-cyclohexanediamine-N′-alanate;
N-2-pyrimidylmethyl-N,N′-bis(2-pyridylmethyl)-isopropylenediamine-N′-alanate;
N-2-pyrimidylmethyl-N,N′-bis(2-pyridylmethyl)-ethylenediamine-N′-methylalanate;
N-2-pyrimidylmethyl-N,N′-bis(2-pyridylmethyl)-cyclohexanediamine-N′-methylalanate; or
N-2-pyrimidylmethyl-N,N′-bis(2-pyridylmethyl)-isopropylenediamine-N′-methylalanate.

3. The iron or manganese complex according to claim 2, wherein each heteroaryl group in L is independently substituted by methyl, ethyl, methoxy, ethoxy and/or hydroxyl.

4. The iron or manganese complex according to claim 3, wherein the ethylene, cyclohexane or isopropylene group in L is substituted by methyl, ethyl, methoxy, ethoxy and/or hydroxyl.

5. The iron or manganese complex according to claim 1, wherein the coordinating species X is selected from:
H₂O; alkyl-CN, CH₃CN; Cl⁻; O²⁻; OH⁻; OOalkyl⁻; OOH⁻; an oxygen-coordinating terminal oxidant of dihydrogen peroxide in its deprotonated form; an alkyl peroxide; iodosylaryl, iodosylbenzene; hypochlorite; perborate; an amine N-oxide; a trialkylamine N-oxide; morpholine N-oxide; 4-methylmorpholine N-oxide; and pyridine N-oxide.

6. The iron or manganese complex according to claim 1, wherein Y is selected from: RCOO⁻, F⁻, Cl⁻, Br⁻, NO₃⁻, BPh₄⁻, ClO₄⁻, BF₄⁻, PF₆⁻, RSO₃⁻, RSO₄⁻, SO₄²⁻, CF₃SO₃⁻, PO₄³⁻, or RPO₄²⁻, wherein R is selected from H, optionally substituted alkyl, and optionally substituted aryl.

7. The iron or manganese complex according to claim 1, wherein X is iodosylbenzene and Y is ClO₄⁻.

8. The iron or manganese complex according to claim 1, wherein L is N,N,N′-tris(2-pyridylmethyl)ethylendiamine-N′-acetate; X is iodosylbenzene, alkyl-OO⁻, HOO⁻, alkyl-CN, or H₂O; and Y is ClO₄⁻.

9. A process for the preparation of an iron or manganese complex having general formula (III): [LM(X)]^{z}Y_{q}
wherein
M represents a metal ion selected from Fe or Mn in the II, III, IV or V oxidation state;
L represents a monoanionic potentially hexadentate ligand containing one carboxylate group and five nitrogen donor atoms, which ligand coordinates to the Fe or Mn metal ion in the complex via said carboxylate group and at least four of said nitrogen donor atoms and has the general formula (IV):
R¹R¹N-A-N(R¹)WC(O)O⁻ (IV)
wherein:
each R¹ independently represents R²—B, in which R² represents an optionally substituted group selected from the group consisting of alkylene, alkenylene, oxyalkylene, aminoalkylene and alkylene ether, and B represents an optionally substituted heteroaryl group selected from the group consisting of pyridinyl, pyrazinyl, pyrazolyl, imidazolyl, benzimidazolyl, pyrimidinyl, triazolyl and thiazolyl;
A represents an optionally substituted alkylene or arylene bridging group selected from the group consisting of CH₂CH₂—, —CH₂CH₂CH₂—, —C₆H₁₀—, and C₆H₄; and
W represents a group selected from the group consisting of alkylene, arylene and arylalkylene;
X represents a coordinating species selected from the group of oxygen-coordinating terminal oxidants including peroxides, peracids, iodosylaryls, hypohalides, oxone, N-oxides and dioxygen;
Y represents a non-coordinating counter ion;
z represents the charge of the complex and is an integer which can be positive or zero; and
q=z/[charge Y]
wherein the process comprises mixing an iron or manganese complex having general formula (I) [(LH)₂Fe₂(O)]^{z}Y_{q} or (II) [LM]^{z}Y_{q} with an oxidant in an organic solvent to form a solution, wherein the oxidant is selected from peroxides, peracids, iodosylaryls, hypohalides, oxone, N-oxides or dioxygen.

10. The process according to claim 9 further comprising precipitating [LM(X)]^{z}Y_{q} from the solution by adding an appropriate solvent.

11. The process according to claim 9, wherein L is selected from:
N,N,N′-tris(2-pyridylmethyl)ethylenediamine-N′-acetate;
N,N,N′-tris(2-pyridylmethyl)cyclohexanediamine-N′-acetate;
N,N,N′-tris(2-pyridylmethyl)isopropylenediamine-N′-acetate;
N,N,N′-tris(2-pyridylmethyl)ethylenediamine-N′-alanate;
N,N,N′-tris(2-pyridylmethyl)cyclohexanediamine-N′-alanate;
N,N,N′-tris(2-pyridylmethyl)isopropylenediamine-N′-alanate;
N,N,N′-tris(2-pyridylmethyl)ethylenediamine-N′-methylalanate;
N,N,N′-tris(2-pyridylmethyl)cyclohexanediamine-N′-methylalanate;
N,N,N′-tris(2-pyridylmethyl)isopropylenediamine-N′-methylalanate;
N,N,N′-tris(1-pyrazolylmethyl)ethylenediamine-N′-acetate;
N,N,N′-tris(1-pyrazolylmethyl)cyclohexanediamine-N′-acetate;
N,N,N′-tris(1-pyrazolylmethyl)isopropylenediamine-N′-acetate;
N,N,N′-tris(1-pyrazolylmethyl)ethylenediamine-N′-alanate;
N,N,N′-tris(1-pyrazolylmethyl)cyclohexanediamine-N′-alanate;
N,N,N′-tris(1-pyrazolylmethyl)isopropylenediamine-N′-alanate;
N,N,N′-tris(1-pyrazolylmethyl)ethylenediamine-N′-methylalanate;
N,N,N′-tris(1-pyrazolylmethyl)cyclohexanediamine-N′-methylalanate;
N,N,N′-tris(1-pyrazolylmethyl)isopropylenediamine-N′-methylalanate;
N,N,N′-tris(2-imidazolylmethyl)ethylenediamine-N′-acetate;
N,N,N′-tris(2-imidazolylmethyl)cyclohexanediamine-N′-acetate;
N,N,N′-tris(2-imidazolylmethyl)isopropylenediamine-N′-acetate;
N,N,N′-tris(2-imidazolylmethyl)ethylenediamine-N′-alanate;
N,N,N′-tris(2-imidazolylmethyl)cyclohexanediamine-N′-alanate;
N,N,N′-tris(2-imidazolylmethyl)isopropylenediamine-N′-alanate;
N,N,N′-tris(2-imidazolylmethyl)ethylenediamine-N′-methylalanate;
N,N,N′-tris(2-imidazolylmethyl)cyclohexanediamine-N′-methylalanate;
N,N,N′-tris(2-imidazolylmethyl)isopropylenediamine-N′-methylalanate;
N,N,N′-tris(2-pyrimidylmethyl)ethylenediamine-N′-acetate;
N,N,N′-tris(2-pyrimidylmethyl)ethylenediamine-N′-acetate;
N,N,N′-tris(2-pyrimidylmethyl)cyclohexanediamine-N′-acetate;
N,N,N′-tris(2-pyrimidylmethyl)isopropylenediamine-N′-acetate;
N,N,N′-tris(2-pyrimidylmethyl)ethylenediamine-N′-alanate;
N,N,N′-tris(2-pyrimidylmethyl)cyclohexanediamine-N′-alanate;
N,N,N′-tris(2-pyrimidylmethyl)isopropylenediamine-N′-alanate;
N,N,N′-tris(2-pyrimidylmethyl)ethylenediamine-N′-methylalanate;
N,N,N′-tris(2-pyrimidylmethyl)cyclohexanediamine-N′-methylalanate;
N,N,N′-tris(2-pyrimidylmethyl)isopropylenediamine-N′-methylalanate;
N,N,N′-tris(1-1,2,4-triazolylmethyl)ethylenediamine-N′-acetate;
N,N,N′-tris(1-1,2,4-triazolylmethyl)cyclohexanediamine-N′-acetate;
N,N,N′-tris(1-1,2,4-triazolylmethyl)isopropylenediamine-N′-acetate;
N,N,N′-tris(1-1,2,4-triazolylmethyl)ethylenediamine-N′-alanate;
N,N,N′-tris(1-1,2,4-triazolylmethyl)cyclohexanediamine-N′-alanate;
N,N,N′-tris(1-1,2,4-triazolylmethyl)isopropylenediamine-N′-alanate;
N,N,N′-tris(1-1,2,4-triazolylmethyl)ethylenediamine-N′-methylalanate;
N,N,N′-tris(1-1,2,4-triazolylmethyl)cyclohexanediamine-N′-methylalanate;
N,N,N′-tris(1-1,2,4-triazolylmethyl)isopropylenediamine-N′-methylalanate;
N,N-bis(2-pyridylmethyl)ethylenediamine-N′-1-pyrazolylmethyl-N′-acetate;
N,N-bis(2-pyridylmethyl)cyclohexanediamine-N′-1-pyrazolylmethyl-N′-acetate;
N,N-bis(2-pyridylmethyl)isopropylenediamine-N′-1-pyrazolylmethyl-N′-acetate;
N,N-bis(2-pyridylmethyl)ethylenediamine-N′-1-pyrazolylmethyl-N′-alanate;
N,N-bis(2-pyridylmethyl)isopropylenediamine-N′-1-pyrazolylmethyl-N′-alanate;
N,N-bis(2-pyridylmethyl)isopropylenediamine-N′-1-pyrazolylmethyl-N′-alanate;
N,N-bis(2-pyridylmethyl)ethylenediamine-N′-1-pyrazolylmethyl-N′-methylalanate;
N,N-bis(2-pyridylmethyl)isopropylenediamine-N′-1-pyrazolylmethyl-N′-methylalanate;
N,N-bis(2-pyridylmethyl)isopropylenediamine-N′-1-pyrazolylmethyl-N′-methylalanate;
N,N-bis(2-pyridylmethyl)ethylenediamine-N′-2-imidazolylmethyl-N′-acetate;
N,N-bis(2-pyridylmethyl)cyclohexanediamine-N′-2-imidazolylmethyl-N′-acetate;
N,N-bis(2-pyridylmethyl)isopropylenediamine-N′-2-imidazolylmethyl-N′-acetate;
N,N-bis(2-pyridylmethyl)ethylenediamine-N′-1-2-imidazolylmethyl-N′-alanate;
N,N-bis(2-pyridylmethyl)cyclohexanediamine-N′-1-2-imidazolylmethyl-N′-alanate;
N,N-bis(2-pyridylmethyl)isopropylenediamine-N′-1-2-imidazolylmethyl-N′-alanate;
N,N-bis(2-pyridylmethyl)ethylenediamine-N′-1-2-imidazolylmethyl-N′-methylalanate;
N,N-bis(2-pyridylmethyl)cyclohexanediamine-N′-1-2-imidazolylmethyl-N′-methylalanate;
N,N-bis(2-pyridylmethyl)isopropylenediamine-N′-1-2-imidazolylmethyl-N′-methylalanate;
N,N-bis(2-pyridylmethyl)ethylenediamine-N′-2-pyrimidylmethyl-N′-acetate;
N,N-bis(2-pyridylmethyl)cyclohexanediamine-N′-2-pyrimidylmethyl-N′-acetate;
N,N-bis(2-pyridylmethyl)isopropylenediamine-N′-2-pyrimidylmethyl-N′-acetate;
N,N-bis(2-pyridylmethyl)ethylenediamine-N′-2-pyrimidylmethyl-N′-alanate;
N,N-bis(2-pyridylmethyl)cyclohexanediamine-N′-2-pyrimidylmethyl-N′-alanate;
N,N-bis(2-pyridylmethyl)isopropylenediamine-N′-2-pyrimidylmethyl-N′-alanate;
N,N-bis(2-pyridylmethyl)ethylenediamine-N′-2-pyrimidylmethyl-N′-methylalanate;
N,N-bis(2-pyridylmethyl)cyclohexanediamine-N′-2-pyrimidylmethyl-N′-methylalanate;
N,N-bis(2-pyridylmethyl)isopropylenediamine-N′-2-pyrimidylmethyl-N′-methylalanate;
N,N-bis(2-pyridylmethyl)ethylenediamine-N′-1-1,2,4-triazolylmethyl-N′-acetate;
N,N-bis(2-pyridylmethyl)cyclohexanediamine-N′-1,2,4-triazolylmethyl-N′-acetate;
N,N-bis(2-pyridylmethyl)isopropylenediamine-N′-1,2,4-triazolylmethyl-N′-acetate;
N,N-bis(2-pyridylmethyl)ethylenediamine-N′-1,2,4-triazolylmethyl-N′-alanate;
N,N-bis(2-pyridylmethyl)cyclohexanediamine-N′-1,2,4-triazolylmethyl-N′-alanate;
N,N-bis(2-pyridylmethyl)isopropylenediamine-N′-1,2,4-triazolylmethyl-N′-alanate;
N,N-bis(2-pyridylmethyl)ethylenediamine-N′-1,2,4-triazolylmethyl-N′-methylalanate;
N,N-bis(2-pyridylmethyl)cyclohexanediamine-N′-1,2,4-triazolylmethyl-N′-methylalanate;
N,N-bis(2-pyridylmethyl)isopropylenediamine-N′-1,2,4-triazolylmethyl-N′—N,N-bis(1-pyrazolylmethyl)ethylenediamine-N′-2-pyridylmethyl-N′-acetate;
N,N-bis(1-pyrazolylmethyl)cyclohexanediamine-N′-2-pyridylmethyl-N′-acetate;
N,N-bis(1-pyrazolylmethyl)isopropylenediamine-N′-2-pyridylmethyl-N′-acetate;
N,N-bis(1-pyrazolylmethyl)ethylendiamine-N′2-pyridylmethyl-N′-alanate;
N,N-bis(1-pyrazolylmethyl)cyclohexanediamine-N′-2-pyridylmethyl-N′-alanate;
N,N-bis(1-pyrazolylmethyl)isopropylenediamine-N′-2-pyridylmethyl-N′-alanate;
N,N-bis(1-pyrazolylmethyl)ethylendiamine-N′-2-pyridylmethyl-N′-methylalanate;
N,N-bis(1-pyrazolylmethyl)cyclohexanediamine-N′-2-pyridylmethyl-N′-methylalanate;
N,N-bis(1-pyrazolylmethyl)isopropylenediamine-N′-2-pyridylmethyl-N′-methylalanate;
N,N-bis(2-imidazolylmethyl)ethylenediamine-N′-2-pyridylmethyl-N′-acetate;
N,N-bis(2-imidazolylmethyl)cyclohexanediamine-N′2-pyridylmethyl-N′-acetate;
N,N-bis(2-imidazolylmethyl)isopropylenediamine-N′-2-pyridylmethyl-N′-acetate;
N,N-bis(2-imidazolylmethyl)ethylendiamine-N′2-pyridylmethyl-N′-alanate;
N,N-bis(2-imidazolylmethyl)cyclohexanediamine-N′-2-pyridylmethyl-N′-alanate;
N,N-bis(2-imidazolylmethyl)isopropylenediamine-N′-2-pyridylmethyl-N′-alanate;
N,N-bis(2-imidazolylmethyl)ethylendiamine-N′-2-pyridylmethyl-N′-methylalanate;
N,N-bis(2-imidazolylmethyl)cyclohexanediamine-N′2-pyridylmethyl-N′-methylalanate;
N,N-bis(2-imidazolylmethyl)isopropylenediamine-N′-2-pyridylmethyl-N′-methylalanate;
N,N-bis(2-pyrimidylmethyl)ethylenediamine-N′-2-pyridylmethyl-N′-acetate;
N,N-bis(2-pyrimidylmethyl)cyclohexanediamine-N′-2-pyridylmethyl-N′-acetate;
N,N-bis(2-pyrimidylmethyl)isopropylenediamine-N′-2-pyridylmethyl-N′-acetate;
N,N-bis(2-pyrimidylmethyl)ethylendiamine-N′-2-pyridylmethyl-N′-alanate;
N,N-bis(2-pyrimidylmethyl)cyclohexanediamine-N′-2-pyridylmethyl-N′-alanate;
N,N-bis(2-pyrimidylmethyl)isopropylenediamine-N′-2-pyridylmethyl-N′-alanate;
N,N-bis(2-pyrimidylmethyl)ethylendiamine-N′-2-pyridylmethyl-N′-methylalanate;
N,N-bis(2-pyrimidylmethyl)cyclohexanediamine-N′-2-pyridylmethyl-N′-methylalanate;
N,N-bis(2-pyrimidylmethyl)isopropylenediamine-N′-2-pyridylmethyl-N′-methylalanate;
N,N-bis(1-1,2,4-triazolylmethyl)ethylenediamine-N′-2-pyridylmethyl-N′-acetate;
N,N-bis(1-1,2,4-triazolylmethyl)cyclohexanediamine-N′-2-pyridylmethyl-N′-acetate;
N,N-bis(1-1,2,4-triazolylmethyl)isopropylenediamine-N′-2-pyridylmethyl-N′-acetate;
N,N-bis(1-1,2,4-triazolylmethyl)ethylendiamine-N′-2-pyridylmethyl-N′-alanate;
N,N-bis(1-1,2,4-triazolylmethyl)cyclohexanediamine-N′-2-pyridylmethyl-N′-alanate;
N,N-bis(1-1,2,4-triazolylmethyl)isopropylenediamine-N′-2-pyridylmethyl-N′-alanate;
N,N-bis(1-1,2,4-triazolylmethyl)ethylendiamine-N′-2-pyridylmethyl-N′-methylalanate;
N,N-bis(1-1,2,4-triazolylmethyl)cyclohexanediamine-N′-2-pyridylmethyl-N′-methylalanate;
N,N-bis(1-1,2,4-triazolylmethyl)isopropylenediamine-N′-2-pyridylmethyl-N′-methylalanate;
N-1-pyrazolylmethyl-N,N′-bis(2-pyridylmethyl)-ethylenediamine-N′-acetate;
N-1-pyrazolylmethyl-N,N′-bis(2-pyridylmethyl)-cyclohexanediamine-N′-acetate;
N-1-pyrazolylmethyl-N,N′-bis(2-pyridylmethyl)-isopropylenediamine-N′-acetate;
N-1-pyrazolylmethyl-N,N′-bis(2-pyridylmethyl)-ethylenediamine-N′-alanate;
N-1-pyrazolylmethyl-N,N′-bis(2-pyridylmethyl)-cyclohexanediamine-N′-alanate;
N-1-pyrazolylmethyl-N,N′-bis(2-pyridylmethyl)-isopropylenediamine-N′-alanate;
N-1-pyrazolylmethyl-N,N′-bis(2-pyridylmethyl)-ethylenediamine-N′-methylalanate;
N-1-pyrazolylmethyl-N,N′-bis(2-pyridylmethyl)-cyclohexanediamine-N′-methylalanate;
N-1-pyrazolylmethyl-N,N′-bis(2-pyridylmethyl)-isopropylenediamine-N′-methylalanate;
N-2-imidazolylmethyl-N,N′-bis(2-pyridylmethyl)-ethylenediamine-N′-acetate;
N-2-imidazolylmethyl-N,N′-bis(2-pyridylmethyl)-cyclohexanediamine-N′-acetate;
N-2-imidazolylmethyl-N,N′-bis(2-pyridylmethyl)-isopropylenediamine-N′-acetate;
N-2-imidazolylmethyl-N,N′-bis(2-pyridylmethyl)-ethylenediamine-N′-alanate;
N-2-imidazolylmethyl-N,N′-bis(2-pyridylmethyl)-cyclohexanediamine-N′-alanate;
N-2-imidazolylmethyl-N,N′-bis(2-pyridylmethyl)-isopropylenediamine-N′-alanate;
N-2-imidazolylmethyl-N,N′-bis(2-pyridylmethyl)-ethylenediamine-N′-methylalanate;
N-2-imidazolylmethyl-N,N′-bis(2-pyridylmethyl)-cyclohexanediamine-N′-methylalanate;
N-2-imidazolylmethyl-N,N′-bis(2-pyridylmethyl)-isopropylenediamine-N′-methylalanate;
N-1-1,2,4-triazolylmethyl-N,N′-bis(2-pyridylmethyl)-ethylenediamine-N′-acetate;
N-1-1,2,4-triazolylmethyl-N,N′-bis(2-pyridylmethyl)-cyclohexanediamine-N′-acetate;
N-1-1,2,4-triazolylmethyl-N,N′-bis(2-pyridylmethyl)-isopropylenediamine-N′-acetate;
N-1-1,2,4-triazolylmethyl-N,N′-bis(2-pyridylmethyl)-ethylenediamine-N′-alanate;
N-1-1,2,4-triazolylmethyl-N,N′-bis(2-pyridylmethyl)-cyclohexanediamine-N′-alanate;
N-1-1,2,4-triazolylmethyl-N,N′-bis(2-pyridylmethyl)-isopropylenediamine-N′-alanate;
N-1-1,2,4-triazolylmethyl-N,N′-bis(2-pyridylmethyl)-ethylenediamine-N′-methylalanate;
N-1-1,2,4-triazolylmethyl-N,N′-bis(2-pyridylmethyl)-cyclohexanediamine-N′-methylalanate;
N-1-1,2,4-triazolylmethyl-N,N′-bis(2-pyridylmethyl)-isopropylenediamine-N′-methylalanate;
N-2-pyrimidylmethyl-N,N′-bis(2-pyridylmethyl)-ethylenediamine-N′-acetate;
N-2-pyrimidylmethyl-N,N′-bis(2-pyridylmethyl)-cyclohexanediamine-N′-acetate;
N-2-pyrimidylmethyl-N,N′-bis(2-pyridylmethyl)-isopropylenediamine-N′-acetate;
N-2-pyrimidylmethyl-N,N′-bis(2-pyridylmethyl)-ethylenediamine-N′-alanate;
N-2-pyrimidylmethyl-N,N′-bis(2-pyridylmethyl)-cyclohexanediamine-N′-alanate;
N-2-pyrimidylmethyl-N,N′-bis(2-pyridylmethyl)-isopropylenediamine-N′-alanate;
N-2-pyrimidylmethyl-N,N′-bis(2-pyridylmethyl)-ethylenediamine-N′-methylalanate;
N-2-pyrimidylmethyl-N,N′-bis(2-pyridylmethyl)-cyclohexanediamine-N′-methylalanate; or
N-2-pyrimidylmethyl-N,N′-bis(2-pyridylmethyl)-isopropylenediamine-N′-methylalanate.

12. The process according to claim 11, wherein each heteroaryl group in L is independently substituted by methyl, ethyl, methoxy, ethoxy and/or hydroxyl.

13. The process according to claim 12, wherein the ethylene, cyclohexane, or isopropylene group in L is substituted by methyl, ethyl, methoxy, ethoxy and/or hydroxyl.

14. The process according to claim 11, wherein M is Fe; L is N,N,N′-tris(2-pyridylmethyl)ethylendiamine-N′-acetate; X is selected from the group consisting of iodosylbenzene, alkyl-OO⁻, HOO⁻, and O²⁻; and Y is ClO₄⁻.

15. A method for the catalysis of an oxidation reaction, wherein the method comprises combining an iron or manganese complex having formula (I), (II) or (III) according to claim 1 with a compound to be oxidized and an appropriate oxidant in a solution, and after completion of the oxidation reaction, isolating the oxidized product.

16. The method according to claim 15, wherein the oxidation reaction is the oxidation of an organic substrate, and wherein the method comprises combining the iron or manganese complex having formula (I), (II) or (III) with an organic substrate to be oxidized and the appropriate oxidant in an organic solvent.

17. The method according to claim 16, wherein the organic substrate to be oxidized is selected from the group consisting of sulfides; aromatic and non-aromatic alkenes; amines; alkanes; and alcohols.

18. The method according to claim 15, wherein the oxidation reaction is oxidation of water, and wherein the appropriate oxidant is added to an aqueous solution of a manganese complex having formula (II) or (III), wherein the aqueous solution may optionally further include an organic solvent.

19. The method according to claim 18, wherein the oxidant is selected from the group consisting of tert-butyl hydrogen peroxide, (NH₄)₂[Ce(NO₃)₆], Ce(ClO₄)₄, oxone, and photo-oxidation mediated by [Ru(bipyridine)₃]²⁺ in the presence of an electron acceptor.
